(19) United States
(12) Patent Application Publication
(10) Pub. No.: US 2018/0043119 A1
(43) Pub. Date: Feb. 15, 2018

(54) SPEAKING VALVE ASSEMBLIES AND TRACHEOSTOMY TUBE ASSEMBLIES

(71) Applicant: SMITHS MEDICAL INTERNATIONAL LIMITED, Ashford (GB)

(72) Inventors: Timothy Bateman, Hythe (GB); Stephen James Field, Canterbury (GB); Mark Andrew Graham, Folkestone (GB); Andrew Thomas Jeffrey, Hythe (GB); Christopher John Woosnam, Great Sutton (GB)

(73) Assignee: SMITHS MEDICAL INTERNATIONAL LIMITED, Ashford (GB)

(21) Appl. No.: 15/551,054

(22) PCT Filed: Feb. 15, 2016
(86) PCT No.: PCT/GB2016/000032

§ 371 Date: Aug. 15, 2017

(30) Foreign Application Priority Data
Mar. 4, 2015 (GB) 1503693.2
Mar. 27, 2015 (GB) 1505371.3

(51) Int. Cl. A61M 16/04; A61M 16/20

(52) U.S. Cl. CPC A61M 16/0468; A61M 16/209; A61M 2039/248

(57) ABSTRACT

A speaking valve assembly (2, 200) for fitting to the machine end (13) of a tracheostomy tube (1) has a one-way valve (23, 236) that allows inhalation but prevents exhalation. The assembly further includes a pressure relief arrangement that remains closed during normal respiration and vocalisation but opens when pressure in the assembly rises above a normal level. In one arrangement the pressure relief arrangement takes the form of a ball valve (30) with a ball (32) that is lifted away from an opening (31) by excess pressure but falls back to block the opening when pressure reduces. An alternative pressure arrangement has a slidable collar (226) that normally covers vent apertures (223) in a housing (220) of the assembly. When pressure in the assembly rises above a normal level the collar (226) is pushed out to expose the vent apertures (223) and allow air to escape.

This invention relates to speaking valve assemblies of the kind having a patient end adapted for fitting with the machine end of a tracheostomy tube, the assembly having a first valve located towards the machine end of the assembly, the first valve being normally closed but being opened by a reduced pressure at the patient end of the assembly during patient inhalation to allow gas to flow through the first valve to the patient end of the assembly.

Tracheostomy tubes are used to ventilate patients during and after surgery. As the patient begins to recover, it is preferable for him to be gradually weaned off breathing through the tube before it is completely removed. Also, in order to enable the patient to speak it is necessary to allow at least a part of the air exhaled by the patient to flow up past the tracheostomy tube to the vocal folds instead of out through the machine end of the tube. Both these ends can be achieved by deflating or partially deflating the sealing cuff on the tube. Alternatively, a fenestrated tracheostomy tube can be used having one or more small openings in its side wall so that a part of the patient's breathing passes through these openings and via his nose or mouth, instead of through the machine end of the tracheostomy tube. When the patient needs to speak it is common practice to fit a speaking valve to the machine end of the tube. The speaking valve includes a one-way valve that enables air to be inhaled by the patient through the valve but prevents or limits flow out through the valve so that air instead flows to the larynx via the fenestrations or around the outside of a tube with a deflated cuff. Examples of speaking valves are described in, for example, U.S. Pat. No. 4,325,366, GB2164424, GB2214089, GB2313317, EP78685, EP214243, EP18461, DE2505123 and DE3503874.

A problem arises when a speaking valve is used with an unfenestrated tracheostomy tube if the sealing cuff is inadvertently not deflated since there is no path for exhaled air from the patient. This can lead to the patient suffocating. PCT/GB2015/000212 and PCT/GB2015/000224 suggest alternative arrangements by which this problem can be addressed.

It is an object of the present invention to provide an alternative speaking valve assembly and a tracheostomy tube including such a speaking valve assembly.

According to one aspect of the present invention there is provided a speaking valve assembly of the above-specified kind, characterised in that the assembly further includes a pressure relief arrangement located between the first valve and the patient end of the assembly, that the pressure relief arrangement is normally closed to prevent gas escaping through the arrangement but is arranged to open and allow gas to escape when pressure inside the assembly rises above a level experienced during normal exhalation and vocalisation.

The pressure relief device may include a ball valve. The ball valve may include a circular opening in the wall of the valve, a spherical ball arranged to engage and seal the opening and a cage attached with the valve and surrounding the opening and the ball such that the ball can be moved away from the opening by pressure within the valve and falls back onto the opening when pressure falls.

Alternatively, the pressure relief device may include a slidable pressure relief member that is movable by increased pressure within the valve assembly above a level experienced during normal exhalation and vocalisation from a first position in which it covers a vent to a second position in which it reveals the vent to allow gas to escape through the assembly and via the vent. The pressure relief member is preferably arranged to remain in the second position following increased pressure when the pressure falls below the increased pressure. The vent may be provided by a plurality of apertures spaced around a housing of the assembly, the pressure relief member being provided by a cylindrical collar slidable along the housing over the apertures. The apertures may be elongated and inclined at an angle to the axis of the housing. The first valve is preferably mounted with the cylindrical collar. The slidable collar may have an outer surface region that is concealed within the housing in the first position and is visible externally in the second position. The outer surface region is preferably prominently marked to be visible externally of the speaking valve assembly.

According to another aspect of the present invention there is provided a tracheostomy tube assembly including a tracheostomy tube and a speaking valve assembly according to the above one aspect of the present invention fitted on the machine end of the tube.

A tracheostomy tube including two different forms of speaking valve assembly all according to the present invention will now be described, by way of example, with reference to the accompanying drawings, in which:

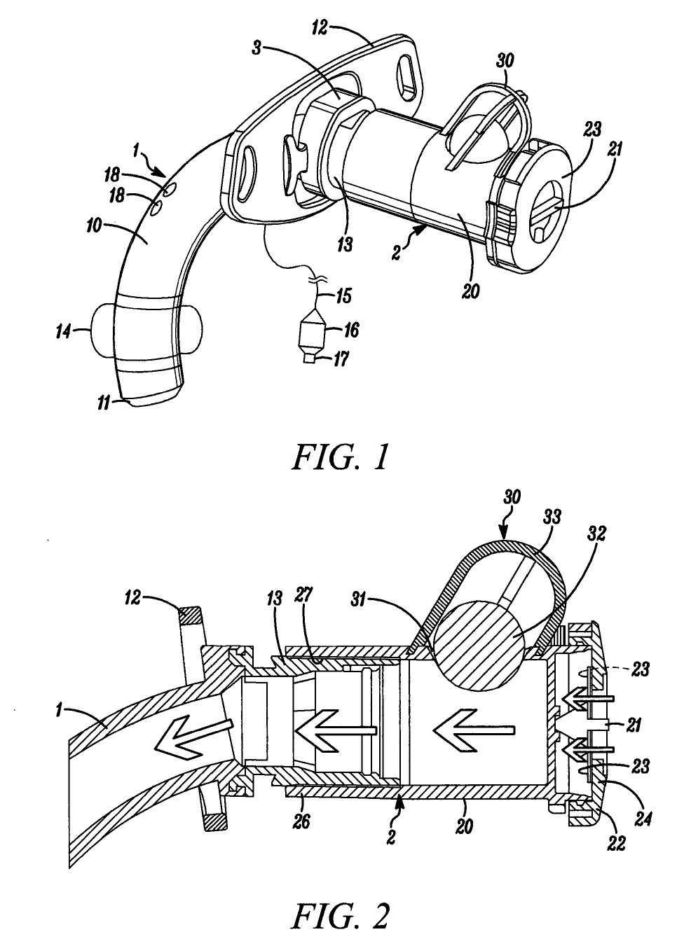

FIG. 1 is a perspective view of the tracheostomy tube and a first form of speaking valve assembly;

FIG. 2 is an enlarged cross-sectional side elevation view of the machine end of the tracheostomy tube and the speaking valve assembly during normal inspiration;

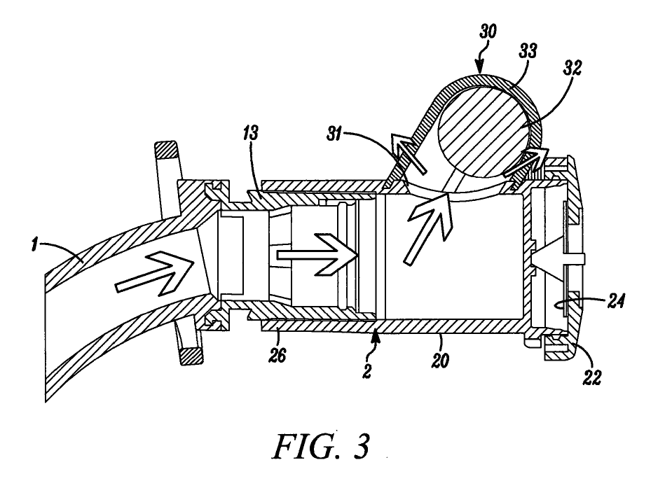

FIG. 3 is an enlarged cross-sectional side elevation view of the machine end of the tracheostomy tube and the speaking valve assembly when normal expiration is prevented;

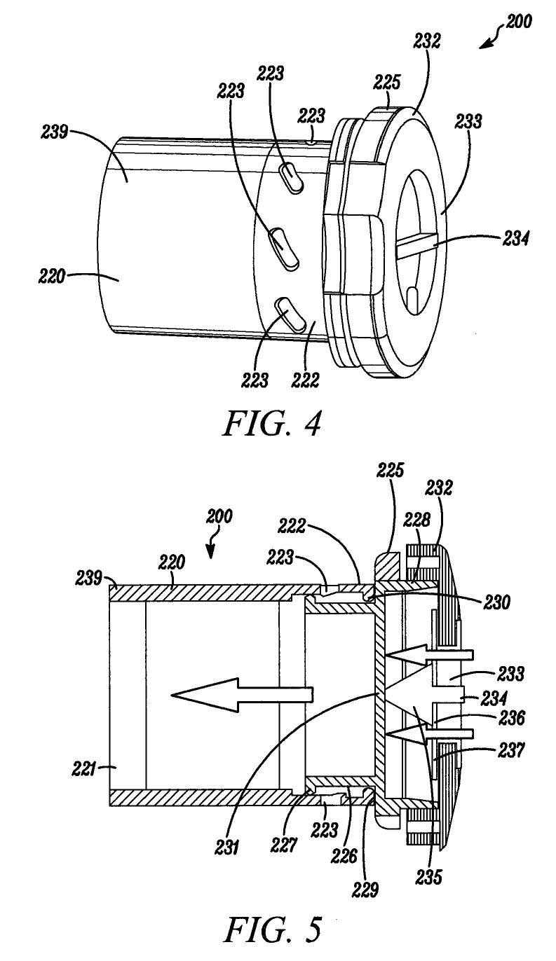

FIG. 4 is an enlarged perspective view of a second, alternative form of speaking valve assembly in its normal condition;

FIG. 5 is an enlarged cross-sectional side elevation view of the second form of speaking valve assembly in its normal condition;

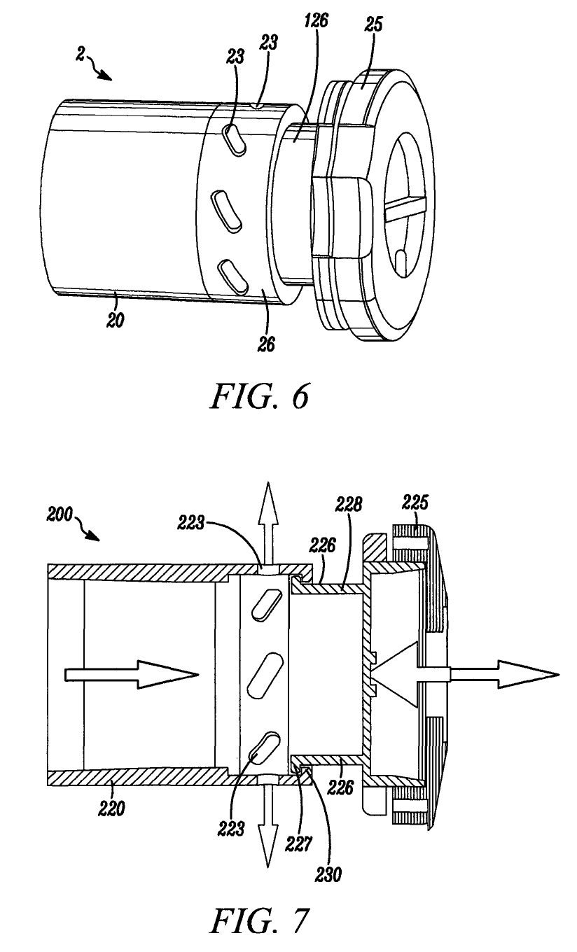

FIG. 6 is an enlarged perspective view of the second form of speaking valve assembly in its venting condition; and

FIG. 7 is an enlarged cross-sectional view of the second form of speaking valve assembly in its venting condition.

With reference first to FIGS. 1 and 2, the assembly comprises a tracheostomy tube 1 and a speaking valve assembly 2 of the first form fitted on the machine end 3 of the tube.

The tracheostomy tube 1 includes a curved shaft 10 of a plastics material and having a circular cross-section. The tube extends from a patient end 11 to a neck flange 12 and a machine end coupling 13 of the conventional 15 mm male tapered kind. A sealing member in the form of an inflatable cuff 14 encircles the shaft 10 towards the patient end 11, the interior of the cuff communicating with an inflation line 15 including an inflation indicator in the form of a pilot balloon 16 and, at its machine end, a sealing valve 17. The tube 1 also has several openings or fenestrations 18 about midway along its length in a location where they are positioned, in use, in the trachea above the sealing cuff 14.

The speaking valve assembly 2 is of cylindrical shape with an outer housing 20. The housing 20 has a support beam 21 extending laterally across its machine end 22 and supporting the centre of a membrane flap valve 23 on the patient side of the beam. The flap valve 23 normally lies flat against an annular sealing seat 24 to prevent any substantial flow of air around the valve during expiration. When the patient inhales, pressure inside the housing 20 falls and causes the membrane 23 to lift at its outer edge away from the seat 24, as shown by the broken line in FIG. 2, and thereby allows air to flow through the valve assembly 2 from its machine end 22 to its patient end 26, and from there, to the patient via the interior of the tube 1. The patient end 26 of the housing 20 has a female tapered internal surface 27 shaped to make a sealing fit on the outside of the machine end coupling 13 of the tracheostomy tube 1.

The speaking valve assembly 2 differs from conventional speaking valves in that it includes a pressure relief device 30 between the flap valve 23 and the patient end 26 of the housing 20. The purpose of the pressure relief device 30 is to allow air to flow out of the speaking valve assembly 2 to atmosphere when pressure rises inside the housing 20 above a level experienced during normal exhalation. In the present example, the pressure relief device 30 takes the form of a ball valve that is normally closed but is opened by pressure above a certain level. The ball valve 30 consists of a circular opening 31 with a frusto-conical profile in the upper side of the wall of the housing 20. A solid, spherical ball 32 having a diameter slightly larger than that of the opening 31 is seated in the opening to seal it closed. A cage 33 is attached to the housing 20 around the opening 31 and the ball 32, being inclined away from the vertical towards the machine end 22 of the housing 20 at an angle of about 30°. The dimensions of the cage 33 are such as to allow the ball 32 to be displaced away from the opening 31 and to fall back into the opening.

During normal respiration, when the tube 1 is used without the speaking valve assembly 2, the fenestrations 18 are closed by an inner cannula (not shown) inserted into the tube from its machine end 3 to extend beyond and cover the fenestrations 18. In this way, air flows into the tube 1 from its machine end 3 and flows out of its patient end 11 during inhalation. During exhalation, air flows in the opposite direction from the patient end 11 and flows out of the machine end 3. When the patient wishes to speak, the inner cannula is removed and the speaking valve assembly 2 is plugged onto the machine end coupling 13 of the tube 1. The speaking valve assembly 2 allows the patient to inhale fairly freely via the flap valve 23. The speaking valve assembly 2, however, prevents normal exhalation via the assembly because the flap valve 23 is closed by elevated pressure in the housing 20 and the mass of the ball 32 is chosen such that the pressure in the housing is insufficient to lift the ball and open the opening 31. Instead, exhaled air flows out of the tube 1 via the fenestrations 18 upwardly along the trachea to the vocal folds so that the patient can speak.

There are, however, situations where a conventional speaking valve assembly could allow the patient to inhale but prevent the patient exhaling. For example, a speaking valve might be fitted to a tube that did not have any fenestrations. This can be done safely if the tube does not have any sealing cuff, since the patient could exhale around the outside of the tube. Alternatively, the valve could be fitted safely to a tube with a sealing cuff providing that the cuff was fully deflated before this was done. It will be appreciated, however, that conventional speaking valve assemblies present a possible hazard if used with unfenestrated tubes if the clinical staff do not ensure the sealing cuff is fully deflated. By contrast, the speaking valve assemblies according to the present invention avoid this problem by providing a safety by-pass path for exhalation gas, such as via the ball valve 30. The ball valve 30, is arranged (as shown in FIG. 3) to be lifted away from sealing engagement with the opening 31 when gas pressure in the housing 20 rises above a level experienced during normal exhalation and vocalisation by an appropriate choice of mass of the ball 32. When the patient inhales again, the pressure inside the housing 20 falls, allowing the ball 32 to fall back by gravity and reseat in the opening 31. This arrangement is advantageous because the alternating movement of the ball 32 in the cage 33 is highly conspicuous and visible to clinicians in the immediate vicinity, giving a clear alarm signal that something is wrong. The rattling noise of the ball 32 moving in the cage 33 enhances the warning. By a suitable selection of hard materials for the ball 32 and cage 33 the noise can be increased.

A second, alternative form of speaking valve assembly 200 will now be described with reference to FIGS. 4 to 7. The speaking valve assembly is of cylindrical shape having an outer housing 220 with a tapered inner surface 221 at its patient end shaped to form a secure fit on the machine end coupling 13 of the tube 1. Towards its opposite, machine end 222 the housing 220 has a ring of several vent apertures or openings 223 extending around the housing. Any number of one or more vent apertures may be used but typically there would be about eight apertures equally spaced from one another around the housing 220. The apertures 223 shown take the form of short, elongated slots with rounded ends inclined at an angle of about 60° to the axis of the housing 220. Apertures of other shapes could be used.

The speaking valve assembly 200 also includes a separate sub-assembly 225 having a collar 226 with an outwardly projecting sealing lip 227 at its patient end. In the normal condition of the valve assembly 200 the collar 226 extends internally of the housing 220 with its sealing lip 227 engaging the inside of the housing on the patient side of the apertures 223 so that these are covered and closed. The vent apertures 223 and the collar 226 together provide a pressure relief arrangement. At its machine end, the sub-assembly 225 is enlarged to form a ring portion 228, the ring portion and collar 226 defining between them an external step 229. The step 229 abuts the end face of an inwardly-extending lip 230 at the machine end of the housing 220, which makes a sliding seal with the outer surface 227 of the collar 226. A cross spar 231 extends diametrically across the sub-assembly 225 level with the step 229. A valve cap 232 is fitted on the outside of the ring portion 228. The valve cap 232 has a central aperture 233 across which a lateral support beam 234 extends. The support beam 234 has a central, forwardly-projecting peg 235 that locates on the rear surface of the spar 231 and supports the centre of a membrane flap valve 236 on the patient side of the beam. The flap valve 236 normally lies flat against an annular sealing seat 237 on the inside of the cap 232 to prevent any substantial flow of air around the valve during expiration. When the patient inhales, pressure inside the assembly 200 falls and causes the membrane 236 to lift at its outer edge away from the seat 237 and thereby allow air to flow through the valve assembly 200 from its machine end 238 to its patient end 239, and from there, to the patient via the interior of the tube 1.

The sub-assembly 225 and vent openings 223 act together as a pressure relief device between the flap valve 236 and the patient end 239 of the housing 220. If expiratory pressure inside the speaking valve assembly 200 should rise above a level experienced during normal exhalation and vocalisation, the flap valve 236 would be forced closed and pressure would rise sufficiently to force the sub-assembly 225 to be moved outwardly relative to the housing 220 as shown in FIGS. 6 and 7. As soon as the lip 227 at the patient end of the collar 226 starts to move over the vent apertures 223 exhaled air starts to escape from inside the housing 220 to atmosphere. The inclined shape of apertures 223 causes them to be exposed only gradually by the lip 227, leading to a less sudden drop in pressure within the housing. The pressure needed to open the vent openings 223 are closed by an appropriate choice of friction between the sub-assembly 225 and the housing 220. The sub-assembly 225 remains in the extended, venting position because of the friction between the sub-assembly and the housing 220. In this position the patient can both exhale and inhale freely through the vent openings 223. The outer surface 228 of the collar 226, which is normally concealed within the housing 220 in the retracted position, is marked in a conspicuous manner, such as by being coloured bright red. This arrangement ensures that clinical staff immediately become aware that the speaking valve 200 has been moved to its extended state and that the patient is having difficulties exhaling. When the patient inhales again, the pressure inside the housing 220 falls but the sub-assembly 225 remains extended so as to give a warning to clinical staff that there is a problem.

It is not essential that the pressure relief device be provided by one of the two arrangements described above. Instead, alternative relief valves could be used, such as those including a valve element actuated by a spring rather than relying on the gravity force acting on the mass of a ball.

1-11. (canceled)

12. A speaking valve assembly having a patient end adapted for fitting with the machine end of a tracheostomy tube, the assembly having a first valve located towards the machine end of the assembly, the first valve being normally closed but being opened by a reduced pressure at the patient end of the assembly during patient inhalation to allow gas to flow through the first valve to the patient end of the assembly, characterised in that the assembly further includes a pressure relief arrangement located between the first valve and the patient end of the assembly, that the pressure relief arrangement is normally closed to prevent gas escaping through the arrangement but is arranged to open and allow gas to escape when pressure inside the assembly rises above a level experienced during normal exhalation and vocalisation.

13. A speaking valve assembly according to claim 12, characterised in that the pressure relief device includes a ball valve.

14. A speaking valve assembly according to claim 13, characterised in that the ball valve includes a circular opening in the wall of the valve, a spherical ball arranged to engage and seal the opening and a cage attached with the valve and surrounding the opening and the ball such that the ball can be moved away from the opening by pressure within the valve and falls back onto the opening when pressure falls.

15. A speaking valve assembly according to claim 12, characterised in that the pressure relief device includes a slidable pressure relief member that is movable by increased pressure within the valve assembly above a level experienced during normal exhalation and vocalisation from a first position in which it covers a vent to a second position in which it reveals the vent to allow gas to escape through the assembly and via the vent.

16. A speaking valve assembly according to claim 15, characterised in that the pressure relief member is arranged to remain in the second position following increased pressure when the pressure falls below the increased pressure.

17. A speaking valve assembly according to claim 15, characterised in that the vent is provided by a plurality of apertures spaced around a housing of the assembly, and that the pressure relief member is provided by a cylindrical collar slidable along the housing over the apertures.

18. A speaking valve assembly according to claim 17, characterised in that the apertures are elongated and are inclined at an angle to the axis of the housing.

19. A speaking valve assembly according to claim 17, characterised in that the first valve is mounted with the cylindrical collar.

20. A speaking valve assembly according to claim 17, characterised in that the slidable collar has an outer surface region that is concealed within the housing in the first position and is visible externally in the second position.

21. A speaking valve assembly according to claim 20, characterised in that the outer surface region is prominently marked to be visible externally of the speaking valve assembly.

22. A tracheostomy tube assembly including a tracheostomy tube and a speaking valve assembly having a patient end fitted on a machine end of the tracheostomy tube, the assembly having a first valve located towards the machine end of the assembly, the first valve being normally closed but being opened by a reduced pressure at the patient end of the assembly during patient inhalation to allow gas to flow through the first valve to the patient end of the assembly, wherein the assembly further includes a pressure relief arrangement located between the first valve and the patient end of the assembly, that the pressure relief arrangement is normally closed to prevent gas escaping through the arrangement but is arranged to open and allow gas to escape when pressure inside the assembly rises above a level experienced during normal exhalation and vocalisation.
